(19) United States
(12) Patent Application Publication
(10) Pub. No.: US 2010/0040713 A1
(43) Pub. Date: Feb. 18, 2010

(54) FAT ABSORPTION INHIBITOR AND FOOD AND DRINK USING THE SAME

(75) Inventors: Hisashi Kataoka, Tokyo (JP); Tomoko Shiromizu, Tokyo (JP); Kenji Kawasaki, Tokyo (JP)

Correspondence Address: BIRCH STEWART KOLASCH & BIRCH, PO BOX 747, FALLS CHURCH, VA, 22040-0747, US

(73) Assignee: KRACIE FOODS, LTD., Tokyo (JP)

(21) Appl. No.: 12/377,233

(22) PCT Filed: Aug. 10, 2007
(86) PCT No.: PCT/JP2007/065985

§ 371 Date: Mar. 6, 2009

(30) Foreign Application Priority Data
Aug. 11, 2006 (JP) 2006-220710
Feb. 8, 2007 (JP) 2007-028811

(51) Int. Cl. A61K 36/00

(52) U.S. Cl. 424/776

(57) ABSTRACT

The present invention relates to a fat absorption inhibitor containing a chestnut skin extract, and a food and drink containing that fat absorption inhibitor. The present invention can therefore provide a fat absorption inhibitor, and a food and drink using the same, that can be obtained from waste products following primary use, inhibit increases in lipids such as blood neutral fats by inhibiting absorption of dietary lipids, and in turn are effective in preventing accumulation of lipids, have superior suitability for continuous ingestion without having an impact on flavor, and do not require a complex production process.

### TECHNICAL FIELD

The present invention relates to a fat absorption inhibitor, effective for inhibiting increases in lipids such as neutral fats in the blood by inhibiting absorption of dietary lipids, and in turn preventing accumulation of lipids, and to a food and drink using the same.

### BACKGROUND ART

Since the 1960s in Japan, heart disease (such as myocardial infarction) and cerebrovascular disease (namely, cerebral infarction and stroke) have become the top three causes of death among Japanese, accounting for more than 30% of all deaths. Namely, these diseases are caused by the formation of thrombi in blood vessels in the heart and brain resulting in occlusion of blood vessels and exacerbation of blood flow, thereby oxygen no longer being sent to tissue down the thrombi. In healthy individuals, the body is maintained so that thrombi are not to be formed. However, in cases of lifestyles characterized by a lack of exercise and high-fat diet, excessive amounts of neutral fat and cholesterol are present in the blood, and the accumulation thereof causes hyperlipemia. As a result, blood viscosity increases and blood vessels become narrow and brittle due to fat components adhered to vessel walls, thereby leading to exacerbation of blood flow and embrittlement of vessel walls.

A grape seed extract is known that lowers levels of neutral fats, total cholesterol, low-density lipoprotein cholesterol (hereinafter referred to as LDL cholesterol) in the blood while increasing levels of high-density lipoprotein cholesterol (hereinafter referred to as HDL cholesterol). In general, as one of the factors capable of lowering neutral fats and the like as described above, inhibition of lipase is thought to inhibit absorption of these substances in the intestines or inhibit their re-synthesis into neutral fats (triglycerides) in small intestine mucosa. However, the action mechanism by which grape seeds lower neutral fats is unclear.

Various studies have been conducted in the past on fat absorption inhibitors based on natural food components. For example, Japanese Unexamined Patent Publication No. 2006-191830 (Cited Reference 1) discloses a food containing L-arabinose and a fat absorption inhibitor and/or fat combustion promoter, wherein the fat absorption inhibitor contains catechins. This food is described as allowing the obtaining of effects that inhibit increases in the level of neutral fat in the blood.

In addition, Japanese Unexamined Patent Publication No. 2006-169181 (Cited Reference 2) discloses that a fat absorption inhibitor, containing a dried plant (herb) such as Pulsatilla cernua or an extract thereof extracted and concentrated using 50% ethanol and the like, has lipase inhibitory activity.

Japanese Unexamined Patent Publication No. 2006-193489 (Cited Reference 3) discloses that a body fat accumulation inhibitor, having as an active ingredient thereof a fermentation product obtained by fermenting ginseng, has action of inhibiting increases in blood sugar levels and of ameliorating blood lipid levels.

Japanese Unexamined Patent Publication No. 2005-289951 (Cited Reference 4) discloses that a lipase inhibitor, fat absorption inhibitor or fat accumulation inhibitor containing powdered Eucommia ulmoides leaf, obtained by steaming, drying and crushing the herb Eucommia ulmoides, or an aqueous extract thereof, and a food and drink containing the same, have lipase inhibitory activity and allow the obtaining of effects that lower neutral fat levels in the blood as well as reduce the amount of fatty tissue surrounding reproductive organs.

Japanese Unexamined Patent Publication No. 2005-278478 (Cited Reference 5) discloses that, by ingesting before meals a post-diet rebound preventive drink or fat absorption inhibitor containing a post-fermented tea extract component, absorption of fat from the diet can be inhibited and obesity caused by accumulation of fat in the body can be prevented and ameliorated.

Japanese Unexamined Patent Publication No. 2005-200386 (Cited Reference 6) discloses that a drug containing a polysaccharide modified with a basic group, and a food containing that drug, allow the obtaining of lipase inhibitory effects, fat absorption inhibitory effects and cholesterol absorption inhibitory effects.

In addition, Japanese Unexamined Patent Publication No. H6-321796 (Cited Reference 7) discloses a fat absorption inhibitor containing defatted cacao mass as a main component thereof. Defatted cacao mass, containing high levels of cacao bean dietary fiber, has an action that inhibits absorption of fat in food, and is able to inhibit accumulation of body fat by improving lipid metabolism.

However, the cited references described above have the following problems.

Although Cited References 2, 4 and 5 describe that the fat absorption inhibitors have lipase inhibitory effects as one approach of inhibiting the absorption of fat, there is no demonstration of effects regarding other approaches.

In Cited Reference 1, the combined use of a fat absorption inhibitor such as catechins and L-arabinose is required in order to obtain the effect of inhibiting absorption of fat. Therefore, there is the shortcoming that the fat absorption inhibitory effects of each component alone are low.

Regarding Cited References 1 to 7, the disclosed fat absorption inhibitors have a unique flavor such as a harsh taste, bitter taste or raw smell attributable to the fat absorption inhibitors themselves. Therefore, they have problems of having an impact on the flavor of the foods when contained in foods, while also having the problem of being unsuitable for continuous ingestion.

Cited References 3, 6 and 7 have the problems of requiring special equipment, having to go through special processes and a plurality of steps, and having a complex production process.

In addition, when tea-based raw materials are used in particular, caffeine ends up being contained in large amounts, thereby resulting in the problem of being unsuitable for ingestion before bed or by young persons.

On the other hand, the present applicants found that a chestnut skin extract has lipase inhibitory action, and filed this invention as Japanese Patent Application No. 2006-220709 (Cited Reference 8). However, there is no description in Cited Reference 8 of action with respect to an overall approach to inhibiting absorption of fat.

An object of the present invention is to provide a fat absorption inhibitor that can be obtained from waste products following primary use, that inhibits increases in lipids such as neutral fats in the blood by inhibiting absorption of dietary lipids, is in turn effective for preventing accumulation of lipids, has superior suitability for continuous ingestion without having an impact on flavor, and does not require a complex production process, and to provide a food and drink using the same.

### DISCLOSURE OF THE INVENTION

The inventors of the present invention searched for a material that acts by applying an overall approach to inhibition of lipid absorption, that is also effective for inhibiting the migration of lipids such as neutral fats (triglycerides) ingested in the diet into the blood, and that can be universally used in foods and drinks. The present inventors focused on the chestnut skin extract having lipase inhibitory effects previously found by the present applicants, and conducted extensive studies. As a result, the inventors of the present invention demonstrated that the migration of lipids such as neutral fats (triglycerides) into the blood can be inhibited by a fat absorption inhibitor containing chestnut skin extract, thereby leading to completion of the present invention.

Namely, in order to achieve the object of the present invention, a first aspect of the present invention is a fat absorption inhibitor containing a chestnut skin extract.

In addition, in a second aspect of the present invention, the chestnut skin extract is an extract of cooked chestnut skin. In a third aspect of the present invention, the chestnut skin extract is an extract extracted with an extraction solvent containing a hydrophilic solvent.

Moreover, in a fourth aspect of the present invention, the chestnut skin extract contains chestnut skin tannin. In a fifth aspect of the present invention, the chestnut skin extract contains chestnut skin proanthocyanidine. In a sixth aspect of the present invention, the chestnut skin extract is water-soluble.

In addition, in a seventh aspect of the present invention, the fat absorption inhibitor has the ability to adsorb bile acids. In an eighth aspect of the present invention, the fat absorption inhibitor further has the ability to inhibit lipase.

Finally, a ninth aspect of the present invention is a food and drink containing the fat absorption inhibitor as described above. In particular, in a tenth aspect of the present invention, the food and drink containing the fat absorption inhibitor indicates to the effect that accumulation of lipids is prevented.

### BRIEF DESCRIPTION OF THE DRAWINGS

FIG. 1 is a graph respectively showing time-based changes in blood neutral fat concentrations in rats following administration of olive oil and a fat absorption inhibitor solution or olive oil and water for injection under Japanese Pharmacopoeia (control group) in Experiment 1.

FIG. 2 is a graph respectively showing time-based changes in blood neutral fat concentrations in rats following administration of a fat absorption inhibitor-containing lipid emulsion or the lipid emulsion alone in Experiment 2.

FIG. 3 is a graph respectively showing time-based changes in blood neutral fat concentrations in rats following administration of a fat absorption inhibitor-containing lipid emulsion, an oolong tea FD powder-containing emulsion or the lipid emulsion alone in Experiment 3.

FIG. 4 is a graph respectively showing time-based changes in neutral fat concentrations in humans in a test food ingestion group and a placebo ingestion group in Experiment 5.

FIG. 5 is a graph respectively showing time-based changes in the rate of change of blood chylomicron levels in humans in a test food ingestion group and a placebo ingestion group in Experiment 5.

### BEST MODE FOR CARRYING OUT THE INVENTION

First, the fat absorption inhibitor of the present invention refers to that having action that inhibits the migration of lipids such as neutral fats (triglycerides) into the blood, and the mechanism of that action is presumed to be attributable to inhibition of absorption of lipids in the small intestine by adsorption of bile acids and the like, and inhibition of lipase activity. Namely, said fat absorption inhibitor is thought to inhibit absorption of lipids and reabsorption of bile acids by adsorbing bile acids in the digestive system, resulting in inhibition of re-synthesis of neutral fats in the digestive system and migration of neutral fats into the blood, as well as inhibition of lipid accumulation. Moreover, the fat absorption inhibitor also inhibits lipase activity in the digestive system in addition to the action of adsorbing bile acids, thereby inhibiting decomposition of neutral fats. Accordingly, the fat absorption inhibitor is an extremely effective substance that is thought to synergistically prevent accumulation of lipids.

The fat absorption inhibitor of the present invention contains a chestnut skin extract. There are no particular limitations on the variety or size of chestnuts used as raw materials in the present invention, and may be suitably selected from those typically used. Examples of varieties of chestnuts that can be used include Japanese chestnuts, European chestnuts, Chinese chestnuts, American chestnuts, etc.

For the chestnut skin used in the fat absorption inhibitor of the present invention, the chestnut inner skin or hard outermost chestnut skin obtained by removing the fleshy portion off the seed nut from which the chestnut bur has been removed, are used. These may be suitably selected for use alone or as a combination thereof. The chestnut skin may be a waste product obtained following primary use or may be obtained by peeling from the chestnut with the flesh still intact for special use, but there are no particular limitations thereon. The use of a waste product following primary use is preferable in terms of being inexpensive, enabling a stable supply of raw materials, and reducing the amount of waste products discharged.

The chestnut skin may be used as is, or it may be used after subjecting to various other treatments such as, either alone or in combination, heating (cooking, simmering, steaming, boiling, etc), freezing, and drying. In particular, chestnut skin subjected to cooking treatment is preferably used in terms of preferably demonstrating effects without impairing the inherent flavor of a food even if added in an effective amount, and of being able to impart an aromatic and delicious flavor when added in large amounts, thereby preferably used in drinks such as coffee or tea (and particularly roasted drinks and foods), bakery foods, etc, and of imparting a preferable amber color tone. In addition, chestnut skin subjected to cooking is also preferable in terms of exhibiting little occurrence of putrefaction or degeneration caused by molds and the like during storage. Moreover, extracts using cooked chestnut skin are preferable in terms of demonstrating low levels of residue in the extraction process thereby enabling extracts to be obtained efficiently. When using chestnut skin subjected to cooking treatment, cooking conditions of the chestnut skin consist of, in the case of raw chestnuts for use as peeled chestnuts for example, cooking for about 5 to 10 minutes at 250 to 400° C. by hot-air roasting, but are not necessarily limited thereto.

A chestnut skin extract of the present invention refers to that extracted from the chestnut skin described above using a suitable extraction method. The chestnut skin extract preferably contains tannins extracted in terms of allowing the obtaining of superior fat absorption inhibitory effects.

The aforementioned tannins refer to the generic name of plant-derived compounds having a large number of phenolic hydroxyl groups and demonstrating properties that cause animal hides to be tanned, and are broadly classified into hydrolyzable tannins and condensed tannins. Hydrolyzable tannins refer to those typically having a galloyl group, hexahydroxydiphenoyl group and oxide forms thereof and the like for the polyphenol moiety within a molecule thereof, wherein these moieties are ester-bonded with carbohydrates or cyclic polyalcohols within the molecule. On the other hand, condensed tannins refer to those in which flavans such as catechins are bonded with each other by C—C bonds at the C4-C8 or C4-6 position and the like between molecules resulting in the formation of a dimer or larger polymer, and are classified differently than monomer flavonoids. Among these condensed tannins, those that form anthocyanidine by cleavage of a C—C bond are referred to as proanthocyanidines.

As has been previously described, the chestnut skin extract according to the present invention preferably contains tannins. Among these tannins, a condensed tannin is in particular preferably contained, and proanthocyanidine is more preferably contained in terms of allowing the obtaining of superior fat absorption inhibitory effects.

The fat absorption inhibitor of the present invention contains the aforementioned chestnut skin extract as an active ingredient. The phrase “containing as an active ingredient” refers to the containing of the extract to a degree at which a target function is demonstrated. More specifically, the fat absorption inhibitor preferably contains 50% by weight or more of the chestnut skin extract, more preferably contains 80% by weight or more of the chestnut skin extract based on the total weight of the fat absorption inhibitor in terms of the solid fraction thereof. More preferably, the fat absorption inhibitor is composed only of chestnut skin extract from the view point of allowing the obtaining of superior fat absorption inhibitory effects.

The fat absorption inhibitor of the present invention may contain auxiliary materials, suitably selected within a range that does not impair the object of the present invention. Examples of auxiliary materials include saccharide sweeteners (including monosaccharides such as fructose, glucose, tagatose or arabinose, oligosaccharides such as lactose, oligosaccharide, trehalose or maltose, powdered thick malt syrup, dextrin and polyols), intense sweeteners (such as sucralose, Acesulfame K or stevia), polysaccharides such as starch, oils, dairy products, stabilizers, emulsifiers, flavors, food colors, acidulants, flavoring materials (such as eggs, coffee, tea, cocoa, fruit juice and pulp, yogurt and alcoholic beverages), proteins, dietary fiber, vitamins, minerals, etc, and these may be used alone or as a combination of multiple types thereof.

In addition, there are no particular limitations on the form of the fat absorption inhibitor of the present invention, and examples of forms that can be used include a liquid, powder, granules, paste and various other forms. Among these, a powder or granules are preferable from the viewpoint of stably maintaining fat absorption inhibitory effects for a long period of time.

In addition, the ingested amount of the fat absorption inhibitor of the present invention per single meal in terms of the solid amount of the chestnut skin extract is preferably 50 mg or more, more preferably 100 mg or more, and even more preferably 300 mg or more from the viewpoint of allowing the obtaining of superior fat absorption inhibitory effects. Furthermore, there are no particular limitations on the time of ingestion of the fat absorption inhibitor of the present invention, and examples of ingestion times include before or after meals, concurrent with meals, or the like. Ingesting concurrently with meals is particularly preferable from the viewpoint of allowing the obtaining of fat absorption inhibitory effects efficiently.

Next, the fat absorption inhibitor of the present invention is produced, for example, in the manner indicated below when using chestnut skin in the form of a waste product following primary use. Namely, in the first step, chestnut skin is prepared. At this time, finely crushing the chestnut skin is preferred from the viewpoint of allowing the extract to be extracted efficiently.

Furthermore, in the case of cooking the chestnut skin at the stage of preparing the fat absorption inhibitor of the present invention, although cooking treatment and crushing treatment may be carried out in any order, carrying out cooking treatment first followed by crushing is preferable in terms of efficiency. In addition, inactive components in the form of hydrophilic components may be removed in advance by treating such as by washing the chestnut skin with water or immersing in water and then filtering.

At the same time, an extraction solvent for extracting the chestnut skin is prepared. Examples of the extraction solvent include a hydrophilic solvent, polyvalent alcohol, supercritical carbon dioxide, etc. Although these extraction solvents may be used alone or as a combination of a plurality of type, the extraction solvent preferably contains at least a hydrophilic solvent in terms of obtaining a water-soluble fat absorption inhibitor having superior general applicability to foods and processing adaptability. Examples of the hydrophilic solvent include water, ethanol, methanol, propanol, isopropanol, acetone, butanol, acetonitrile, ethyl acetate, tetrahydrofuran, etc. In addition, examples of the polyvalent alcohol include glycerin, polyglycerin, etc. These may be used alone or in the form of an aqueous solution or dispersion comprising a mixture of a plurality thereof in combination. Among these, ethanol in particular is preferably used in terms of obtaining superior fat absorption inhibitory effects and high applicability when contained in a food or drink. Moreover, in addition to the effects described above, a 20 to 80% by weight aqueous ethanol solution is preferably used in terms of enabling a water-soluble fat absorption inhibitor to be efficiently extracted and enhancing general applicability of the fat absorption inhibitor.

In the next step, a chestnut skin extract is extracted using the chestnut skin and solvent prepared in the manner described above. Examples of extraction methods include a reflux procedure, room temperature immersion, etc. Among these, extraction employing a reflux procedure is preferable in terms of efficiently obtaining an extract having superior fat absorption inhibitory action in a short period of time. In the case of extracting by a reflux procedure using ethanol, the temperature of the extraction solvent when contacting with the chestnut skin for extraction is preferably set to 50° C. or higher, in terms of efficiently obtaining an extract having even more superior fat absorption inhibitory action.

An extract obtained in the manner described above may be purified by a combination of purification procedures using a column and the like as necessary. A combination of purification procedures such that 30% by weight or more of proanthocyanidine in terms of the extract solid fraction is extracted is preferable from the viewpoint of obtaining an extract having superior fat absorption inhibitory action.

Next, an auxiliary material and the like is added to the aforementioned extract as necessary, and the mixture is formed into a desired form by an ordinary method such as freeze-drying, vacuum concentration or spray drying and the like to obtain the fat absorption inhibitor of the present invention. Furthermore, dextrin and the like may be used as a binder when molding into a powder or granules.

The fat absorption inhibitor obtained in this manner can be applied to various types of foods and drinks, as well as can also be applied to pharmaceuticals and general industrial products. Among them, the fat absorption inhibitor is preferably applied to a food or drink in particular in terms of enabling the fat absorption inhibitor to be ingested both conveniently and deliciously and obtaining the fat absorption inhibitory action thereof efficiently and easily. In addition, the case that the fat absorption inhibitor is water-soluble is preferred because there are no limitations on the form of the foods, drinks, pharmaceuticals or general industrial products to which it is applied.

There are no particular limitations on the aforementioned food or drink provided it is able to contain the fat absorption inhibitor, examples of which include confections (such as chewing gum, candy, tablets, chocolate, jelly and the like), frozen confections, starch-based foods such as noodles, powdered foods, drinks (such as soup, coffee, tea, fruit juice, cocoa, alcoholic beverages, jellied drinks, and the like), bakery foods (such as bread, biscuits, and the like), oily foods (such as margarine, shortening, fat spread, and the like), dairy products (such as butter, cream, cheese, and the like), etc. In the case that the fat absorption inhibitor contains an extract of cooked chestnut skin in particular, drinks such as coffee and tea as well as bakery foods are preferred in terms of giving flavor. Furthermore, the fat absorption inhibitor may be added to the food and drink in the present invention at a time of suitably selected stage of the production process according to the characteristics and purpose of each food and drink.

Although the content of the fat absorption inhibitor in the food and drink varies according to the type, purpose and the like of each food and drink, the content of the fat absorption inhibitor in the food and drink is preferably 0.001% by weight or more and more preferably 0.01% by weight or more based on the total weight of the food or drink in terms of the solid fraction of the chestnut skin extract from the viewpoint of allowing the obtaining of superior fat absorption inhibitory effects.

The food and drink of the present invention may contain the aforementioned auxiliary materials suitably selected within a range that does not impair the inherent object thereof in addition to the fat absorption inhibitor.

Next, a drink, for example, is produced in the manner described below as an example of a food and drink of the present invention. Namely, a fat absorption inhibitor is first prepared in the previously described manner. On the other hand, a tea is made by brewing tea leaves such as barley tea. The fat absorption inhibitor and, as necessary, auxiliary materials, are then added into the tea and mixed to obtain the drink containing the fat absorption inhibitor of the present invention.

A statement indicating to the effect that the accumulation of lipids is prevented may be provided on not only the tea drink containing the fat absorption inhibitor obtained in the manner described above, but also the food and drink containing the fat absorption inhibitor of the present invention. Examples of such indications include “For persons concerned about neutral fats”, “For persons concerned about body fat”, “Less accumulation of body fat”, “For persons tending to eat large amounts of deep-fried foods”, “Inhibition of increases in blood neutral fat levels”, “For persons tending to eat large amounts of fatty foods”, “Inhibition of increases in blood neutral fat and cholesterol levels”, “For persons concerned about metabolic syndrome”, etc.

### EXAMPLE

The following provides an exemplary description of the present invention based on examples thereof.

### <Preparation of Fat Absorption Inhibitor>

### Example 1

### High Molecular Weight Fraction of Baked Chestnut Skin Residue

After cooking chestnuts from Hebei Province, China for 7 minutes at 300° C. with a hot air roaster, the chestnut skin (hard skin and inner skin) were peeled off and ground with a coffee mill. This chestnut skin powder and distilled water (20° C.) were placed in a flask and after allowing to stand undisturbed overnight at room temperature, the supernatant portion (hydrophilic inactive component) was removed. Then, 50% by weight aqueous ethanol solution was poured thereto and heated in water bath for 4 hours at 70° C. At this time, in order to prevent evaporation of the ethanol, a Liebig condenser was provided in the upper portion of the flask followed by passing water there through to carry out a reflux procedure. The resulting extract liquid was filtered and separated into a filtrate and residue. The filtrate was concentrated followed by freeze-drying to give a fat absorption inhibitor from chestnut skin extract powder (water-soluble).

The total tannin content of the fat absorption inhibitor obtained from cooked chestnut skin powder extract in this manner was 54% by weight based on the total weight of the powder. In addition, the total proanthocyanidine content was 51% by weight based on the total weight of the powder (as determined according to the vanillin-HCL method).

The aforementioned total tannin content was calculated by combining values obtained according to the vanillin-HCL method, method of Wilson et al. (J. Agric. Food Chem., 38, 1678-1683 (1990), and method of Inoue et al. (Analytical Biochemistry, 169, 363-369 (1988)).

### Example 2

### Chestnut Skin Unpurification or Unfractionation

A raw chestnut skin extract powder (water-soluble) was obtained using the same method as Example 1 with the exception of peeling the chestnut skin (hard outer skin and inner skin) from raw chestnuts from Hebei Province, China and grinding the chestnut skin with a coffee mill.

The total tannin content of the fat absorption inhibitor from the raw chestnut skin extract powder obtained in this manner was 68% by weight based on the total weight of the powder (as determined in the same manner as Example 1). In addition, the total proanthocyanidine content was 65% by weight based on the total weight of the powder (as determined in the same manner as Example 1).

<Fat Absorption Inhibition Test>

The neutral fat absorption inhibitory effects of the fat absorption inhibitors prepared in the manner described above were confirmed according to the method described below.

(Experiment 1)

### (1) Test Animals

Eight-week-old, male Wistar rats (Japan SLC, Inc.), acclimated for 1 week prior to the experiment, were used in the test in groups of eight animals each.

### (2) Preparation of Fat Absorption Inhibitor Solution

The fat absorption inhibitor of Example 1 prepared in the manner described above was dissolved in water for injection under Japanese Pharmacopoeia to a concentration of 200 mg/ml. In addition, water for injection under Japanese Pharmacopoeia not containing the fat absorption inhibitor of Example 1 was prepared for use in a control group.

### (3) Test Procedure

3.5 ml/kg body weight of olive oil and 5.0 ml/kg body weight of the fat absorption inhibitor solution prepared in (2) above or water for injection under Japanese Pharmacopoeia were administered orally by gavage nearly simultaneously to rats fasted for 17 hours.

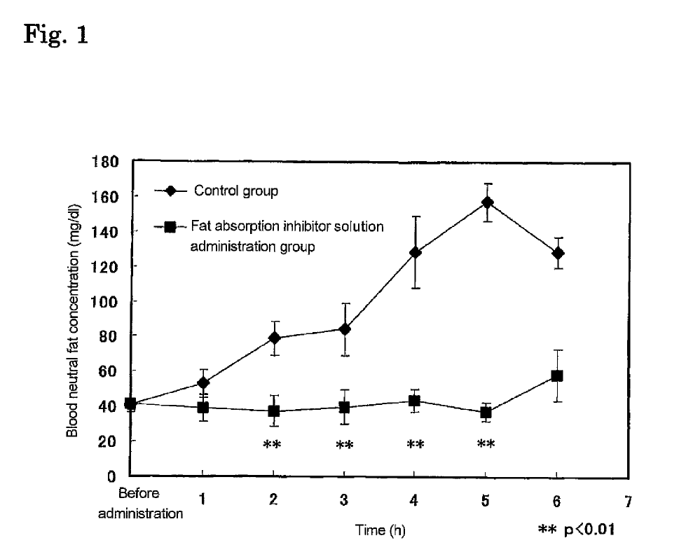

Blood samples were collected from a caudal vein before administration and at 1, 2, 3, 4, 5 and 6 hours after administration followed by measurement of blood neutral fat concentration using the L Type Wako TG H (Wako Pure Chemical Industries, Ltd.). The results are shown in FIG. 1.

As seen from the results of FIG. 1, rats of the control group demonstrated increases in blood neutral fat concentration following administration of olive oil, while rats of the group administered the fat absorption inhibitor solution of Example 1 demonstrated significantly low values of blood neutral fat concentrations.

(Experiment 2)

### (1) Test Animals

Male Wistar rats (Japan SLC, Inc.), acclimated for 1 week prior to the experiment, were used in the test in groups of four animals each.

### (2) Preparation of Lipid Emulsion

A lipid emulsion was prepared in the form of a homogeneous suspension by mixing 6 ml of soybean oil, 6 ml of pure water, 2 g of cholesterol oleate and 100 mg of bile acid followed by ultrasonic treatment.

### (3) Test Procedure

The above lipid emulsion alone or the lipid emulsion containing the fat absorption inhibitor of Example 1 was orally administered in the absence of anesthesia to rats fasted overnight. Here, the lipid emulsion was administered so that each of the dosage of the fat absorption inhibitor was 500 mg/kg body weight or 125 mg/kg body weight.

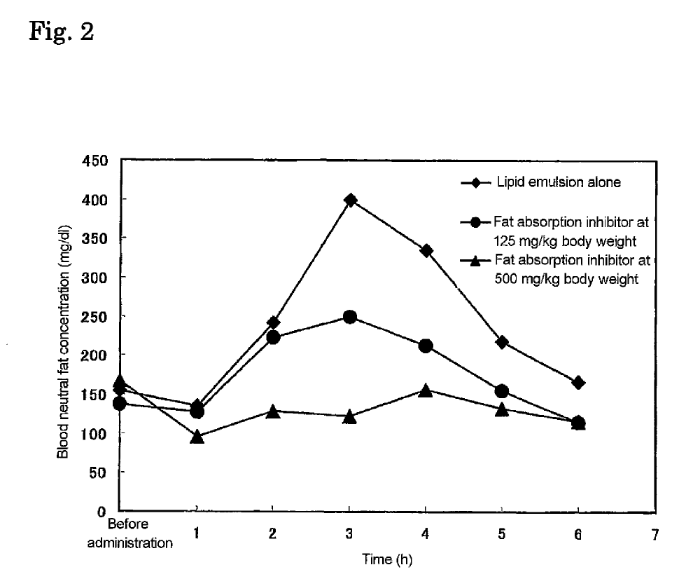

Blood samples were collected from a caudal vein in the absence of anesthesia before administration and at 1, 2, 3, 4, 5 and 6 hours after administration followed by measurement of blood neutral fat concentration using the Triglyceride-E Test Kit (Wako Pure Chemical Industries, Ltd.). The results are shown in FIG. 2.

As seen from the results of FIG. 2, rats administered the lipid emulsion alone demonstrated increases in blood neutral fat concentrations that lasted for 3 hours and then decreased. On the other hand, although rats administered the lipid emulsion containing the fat absorption inhibitor of Example 1 in the dosage of 125 mg/kg body weight demonstrated a similar increasing and decreasing trend in blood neutral fat concentrations, these rats continuously demonstrated lower values of blood neutral fat concentrations than those of rats administered the lipid emulsion alone, and also demonstrated low levels of AUC (area under a blood neutral fat concentration curve for 0 to 6 hours after administration), which is a reflection of the total absorbed amount of neutral fat. In addition, rats administered the lipid emulsion containing the fat absorption inhibitor of Example 1 in the dosage of 500 mg/kg body weight demonstrated no increases in blood neutral fat concentrations from that prior to administration, the values of which were remarkably lower than those of rats administered the lipid emulsion alone, and also demonstrated lower levels of AUC than the case of a dosage of 125 mg/kg body weight.

(Experiment 3)

### (1) Test Animals

### (2) Preparation of Lipid Emulsion

The procedures of steps (1) and (2) were carried out in the same manner as Example 2.

### (3) Test Procedure

The aforementioned lipid emulsion alone, the lipid emulsion containing the fat absorption inhibitor of Example 1, or the lipid emulsion containing a freeze-dried powder of a commercially available oolong tea containing 70 mg/350 ml of oolong tea polymeric polyphenol (hereinafter referred to as oolong tea FD powder) was each orally administered to rats fasted overnight in the absence of anesthesia. Here, the emulsions were administered, respectively, at a dosage of 125 mg/kg body weight of the fat absorption inhibitor, and at a dosage of 388 mg/kg body weight of the oolong tea FD powder, at which is considered that fat absorption inhibitory effects are demonstrated.

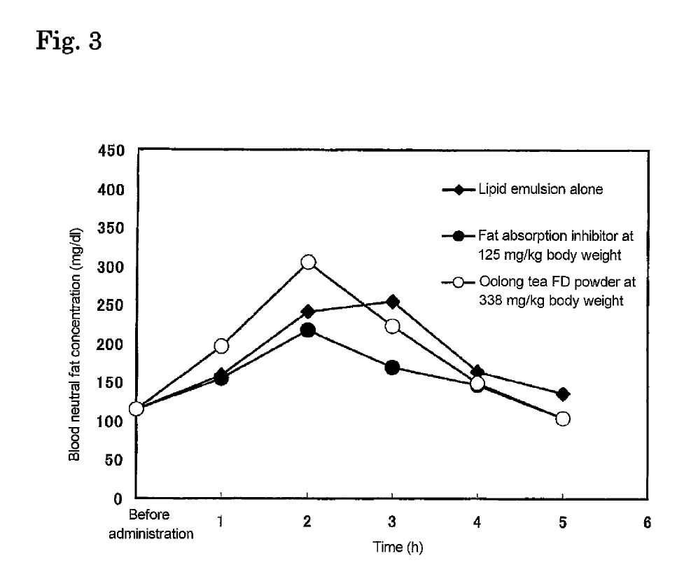

Blood samples were collected from a caudal vein of the rats in the absence of anesthesia before administration and at 1, 2, 3, 4 and 5 hours after administration followed by measurement of blood neutral fat concentration using the Triglyceride-E Test Kit (Wako Pure Chemical Industries, Ltd.). The results are shown in FIG. 3.

As seen from the results of FIG. 3, rats administered the fat absorption inhibitor of Example 1 at 125 mg/kg body weight continuously demonstrated lower values of blood neutral fat concentrations over time in comparison with rats administered the oolong tea FD powder at 388 mg/kg body weight and rats administered the lipid emulsion alone. In addition, the rats administered the fat absorption inhibitor at 125 mg/kg body weight also demonstrated the lowest levels of AUC (area under a blood neutral fat concentration curve for 0 to 5 hours after administration).

Based on the results of Experiments 1 to 3, it can be understood that the fat absorption inhibitor of the present invention lowers blood neutral fat concentrations by a fat absorption inhibitory action, and demonstrate dose-dependent blood neutral fat absorption inhibitory effects. In addition, the gradual slope of the blood neutral fat concentration curves means that there are no sudden fluctuations in neutral fat concentrations in the blood, thereby suggesting that suitable levels of neutral fats in the blood are utilized in the body as nutrients (such as by being transported to fat cells and muscle) without unmanageable excesses of blood neutral fats causing thrombi formation or hyperlipemia, and that the utilization of useful liposoluble components contained in the diet is facilitated.

(Experiment 4)

In addition, the ability of the fat absorption inhibitors of Examples 1 and 2 to adsorb bile acid was confirmed.

### (1) Preparation of Bile Salt Solution

A bile salt (sodium cholate) was dissolved in artificial intestinal fluid to a concentration of 3 mM. The artificial intestinal fluid was prepared by adding 118 ml of a 0.2 mol/l sodium hydroxide test solution to 250 ml of a 0.2 mol/l potassium dihydrogen phosphate test solution and filling up to 1000 ml with pure water in accordance with the Japanese Pharmacopoeia, the Disintegration Test Method, the test solutions, Test Solution 2.

### (2) Test Procedure

10 mg of each of the fat absorption inhibitor of Example 1 or 2, chitosan (Nacalai Tesque Inc.) and α-cellulose (Nacalai Tesque Inc.) were added to 7 ml of the bile salt solution followed by incubating for 60 minutes at 37° C. while shaking. Then the mixture was filtered with a 0.5 μm filter to obtain a test solution. The residual bile salt concentration of the resulting test solution was measured using the Total Bile Acids-Test Wako (Wako Pure Chemical Industries, Ltd.), and the ratio of the reduction in bile salt concentration from the initial concentration of 3 mM was calculated as the adsorption rate of bile salt. The results are shown in Table 1.

TABLE 1

| | Adsorption Rate (%) |
|---|---|
| Example 1 | 88 |
| Example 2 | 86 |
| Chitosan | 32 |
| α-Cellulose | 33 |

As seen from the results of Table 1, the fat absorption inhibitors of the present invention demonstrated a much higher ability to adsorb bile acid than that of chitosan and α-cellulose regarded as having the ability to adsorb bile acid.

Namely, the fat absorption inhibitors of the present invention were demonstrated to have the potential to inhibit absorption of lipids such as neutral fats and dietary cholesterol from the small intestine by adsorbing bile acid in the duodenum and small intestine thereby inhibiting the formation of micelles of lipids and bile acids. Moreover, the possibility of leading to effects resulting in lowering of cholesterol levels in the body was also suggested as a result of inhibiting reabsorption of bile acids.

(Experiment 5)

Next, the effects of ingestion of the fat absorption inhibitor of the present invention by humans were confirmed with a double-blind crossover study.

### (1) Subjects

The subjects of the study consisted of 10 healthy men and women age 20 to under 65.

### (2) Ingestion Method of Test Food and Placebo Food

The test food ingestion group ingested a test food in the form of 300 mg of the fat absorption inhibitor of Example 1 wrapped in a medicinal wafer with water followed immediately after by ingesting a meal load.

The placebo food ingestion group ingested a placebo food in the same manner as the test food ingestion group with the exception of changing the fat absorption inhibitor of Example 1 to a dried powder of barley tea.

Here, the meal load was prepared according to the composition of Table 2 by adding butter and lard to commercially available corn potage soup to a total lipid content of 40 g.

TABLE 2

| | Content (g) | Lipid (g) |
|---|---|---|
| Corn potage soup | 200.0 | 9.2 |
| Butter | 20.0 | 16.6 |
| Lard | 14.2 | 14.2 |
| Total | 234.2 | 40.0 |

### (3) Test Procedure

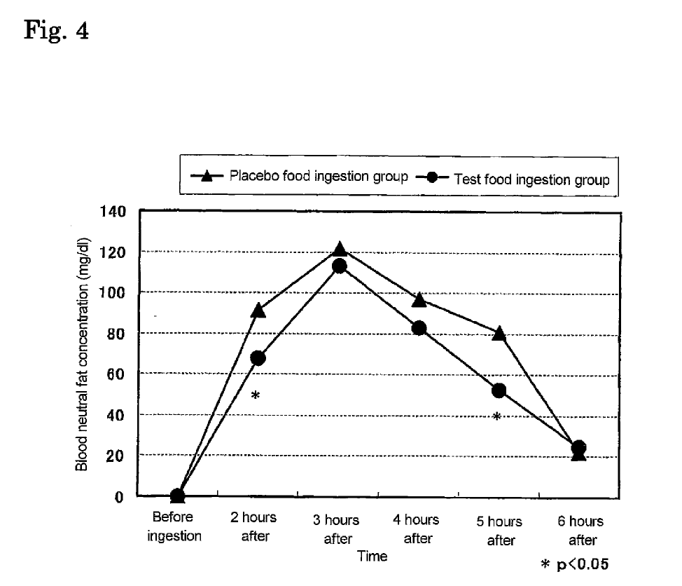

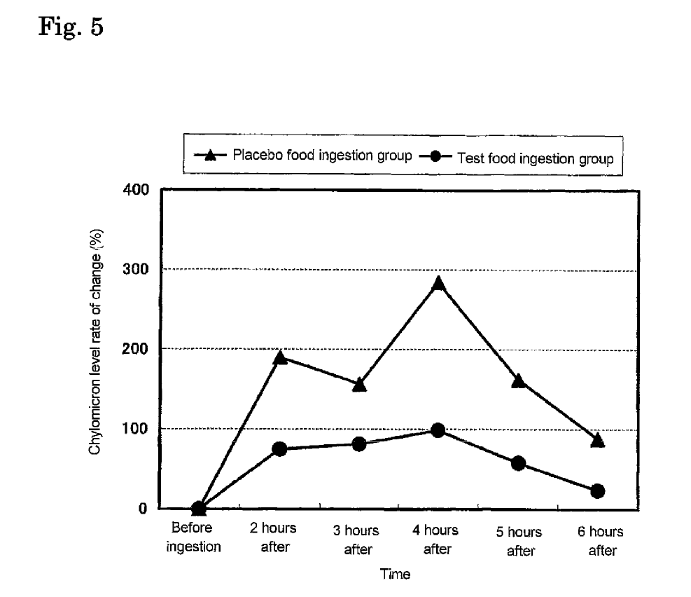

The subjects were divided into two groups of five subjects each. One group was designated as the test food ingestion group and the other as the placebo food ingestion group. Blood samples were collected before ingestion and at 2, 3, 4, 5 and 6 hours after ingestion, followed by measurement of blood neutral fat concentration and blood chylomicron concentration. The blood neutral fat concentrations were measured using a method complying with the Japan Society of Clinical Chemistry (JSCC), while blood chylomicron concentrations were measured by heparin-Ca turbidometric quantitation. Moreover, the same test was carried out by crossing over the ingestion groups after providing a two-week washout period. The results are shown in FIGS. 4 and 5.

As seen from the results of FIGS. 4 and 5, subjects of the test food ingestion group demonstrated lower values for both postprandial blood neutral fat concentrations and blood chylomicron concentrations as compared with the placebo food ingestion group. On the basis of this finding, the fat absorption inhibitor of the present invention was clearly determined to have an effect that inhibits increases in postprandial levels of neutral fats in the blood in humans as well. In addition, the aforementioned experimental result indicating that blood chylomicron levels, which are considered to be an indicator of the absorbed amount of dietary lipids, are inhibited demonstrates that the inhibitory effects on blood neutral fats in this test are attributable to inhibition of absorption in the intestinal tract.

### Example 3

### Drink Preparation

Barley tea contained in a PET bottle was produced in accordance with ordinary methods. The fat absorption inhibitor of Example 1 was added to this barley tea at 0.05% by weight to prepare a fat absorption inhibitor-containing barley tea.

In addition, barley tea not containing fat absorption inhibitor was prepared for use as a control.

Barley tea drinks of Example 3 and the control as described above were ingested by 20 expert panelists in an amount of 200 ml after each meal for a period of one week. As a result, the barley tea of Example 3 was judged to not be inferior to the control in terms of flavor, while more preferably, was judged to be easily ingested on a regular basis as a barley tea drink due to its deep amber color, persistent rich flavor and highly aromatic bouquet. In addition, the fat absorption inhibitor demonstrated superior solubility in the barley tea, was uniformly dispersed, did not precipitate even when filled into a clear PET bottle, and demonstrated a favorable appearance.

### INDUSTRIAL APPLICABILITY

Since the fat absorption inhibitor of the present invention has neutral fat absorption inhibitory action, bile acid adsorptive action, action that inhibits increases in blood chylomicron levels and the like, ingestion thereof makes it possible to inhibit absorption of dietary fat, and in turn, improve lipid metabolism and prevent lipid accumulation, thereby eliminating and preventing obesity.

According to the present invention, a fat absorption inhibitor can be provided that is more suitable for continuous ingestion without having an unpleasant taste as compared with conventional fat absorption inhibitors.

In addition, since potent fat absorption inhibitory effects are obtained, there is no need for concomitant use of other fat absorption inhibitors, deterioration of flavor attributable to other fat absorption inhibitors is prevented, and a wide variation of flavors can be deployed.

Moreover, since there are no limitations on the form of the fat absorption inhibitor of the present invention or on the forms of foods and drinks containing the same, it can be ingested with meals and has a high degree of general applicability. In particular, since chestnut skin extract does not contain caffeine, it is suitable for general use in foods such as caffeine-free beverages regardless of the amount or time ingested.

In addition, in the case the fat absorption inhibitor of the present invention is made to be water-soluble in particular, it can be easily mixed and dissolved into foods and drinks in which it is used, thereby resulting in high degrees of applicability and universality to foods and drinks.

In addition, since chestnut inner skin or hard outermost skin, which are discarded in large amounts during production of chestnut confections such as sweet roasted chestnuts, chestnuts in sweet syrup or marron glace, can be used effectively, there is no need to specially cultivate raw materials, raw materials can be supplied both inexpensively and stably, and this is useful for reducing the amount of discharged waste to consider for impacts on the global environment.

Moreover, since the use of chestnut skin eliminates the need to provide a complex process and since an extract having a fat absorption inhibitory action can be obtained from an extraction process alone, a fat absorption inhibitor can be obtained easily and efficiently in a short period of time.

In addition, since the fat absorption inhibitory action of the present invention lowers blood neutral fat concentration and lowers levels of total cholesterol and LDL cholesterol in the blood while increasing levels of HDL cholesterol, it has the potential for preventing or ameliorating hyperlipemia or demonstrating the effect of preventing dandruff.

1. A fat absorption inhibitor containing a chestnut skin extract.

2. The fat absorption inhibitor according to claim 1, wherein the chestnut skin extract is an extract of a cooked chestnut skin.

3. The fat absorption inhibitor according to claim 1, wherein the chestnut skin extract is an extract extracted with an extraction solvent containing a hydrophilic solvent.

4. The fat absorption inhibitor according to claim 1, wherein the chestnut skin extract contains chestnut skin tannin.

5. The fat absorption inhibitor according to claim 1, wherein the chestnut skin extract contains chestnut skin proanthocyanidine.

6. The fat absorption inhibitor according to claim 1, wherein the chestnut skin extract is water-soluble.

7. The fat absorption inhibitor according to claim 1, having the ability to adsorb bile acids.

8. The fat absorption inhibitor according to claim 7, further having the ability to inhibit lipase.

9. A food and drink containing the fat absorption inhibitor according to claim 1.

10. The food and drink according to claim 9 indicating to the effect that accumulation of lipids is prevented.

11. A method of inhibiting absorption of dietary lipids comprising:
orally administering to a subject a composition suitable for ingestion, wherein said composition comprises chestnut skin extract as an active ingredient in an amount effective to inhibit absorption of dietary lipids in said subject.

12. The method according to claim 11, wherein said composition is in the form of a food, a drink, or a pharmaceutical.

13. The method according to claim 12, wherein said composition is a food.

14. The method according to claim 12, wherein said composition is a drink.

15. The method according to claim 11, wherein the amount of said chestnut skin extract is 50 mg or more per administration.

16. The method of claim 12, wherein said chestnut skin extract is present in an amount of 0.001% by weight or more of the total composition.

17. The method of claim 12, wherein said chestnut skin extract is an extract of a cooked chestnut skin.

18. The method of claim 11, wherein said composition is administered in an amount sufficient to inhibit accumulation of lipids in the blood of said subject.
